(19) United States
(12) Patent Application Publication
(10) Pub. No.: US 2009/0141237 A1
(43) Pub. Date: Jun. 4, 2009

(54) Integrated Optical Coherence Imaging Systems for Use in Ophthalmic Applications and Related Methods and Computer Program Products

(75) Inventors: Joseph A. Izatt, Raleigh, NC (US); Michael E. Sullivan, Raleigh, NC (US); Seungbum Woo, Bridgewater, NJ (US); Robert H. Hart, Cary, NC (US); Eric Buckland, Hickory, NC (US)

Correspondence Address: MYERS BIGEL SIBLEY & SAJOVEC, PO BOX 37428, RALEIGH, NC, 27627, US

(73) Assignee: BIOPTIGEN, INC.

(21) Appl. No.: 12/262,692

(22) Filed: Oct. 31, 2008

Related U.S. Application Data
(60) Provisional application No. 60/984,894, filed on Nov. 2, 2007.

(51) Int. Cl. A61B 3/10; G01B 11/02

(52) U.S. Cl. 351/211; 356/497

(57) ABSTRACT

A system for imaging a sample is provided that includes an optical coherence tomography (OCT) portion having an associated OCT path. A second imaging portion is provided having an associated second image path, different from the OCT path. A fixation target having an associated fixation path, different from the second image path and the OCT path is also provided. The OCT path, the second image path and the fixation path are confocal at a common image plane. Related handheld systems are also provided.

### CLAIM OF PRIORITY

The present application claims priority from U.S. Provisional Application No. 60/984,894; (Attorney Docket No. 9526-15PR), filed Nov. 2, 2007, the disclosure of which is hereby incorporated herein by reference as if set forth in its entirety.

### FIELD OF THE INVENTION

The present invention relates to imaging and, more particularly, to optical coherence tomography (OCT) and related systems, methods and computer program products.

### BACKGROUND OF THE INVENTION

Visual field testing is a conventional clinical method utilized in the diagnosis of eye diseases that cause degradation of vision sensitivity. One method utilized in the diagnosis of such diseases is the Standard Automated Perimeter (SAP) test, which tests brightness contrast sensitivity over a large visual field. There are many instruments for performing an SAP test routinely used in clinics including, for example, those produced by Carl Zeiss Meditec (Dublin, Calif.).

Typically, visual field testing utilizes functional field testing techniques. However, a functional field test technique is a functional test of vision degradation. Due to the human eye's complex multiplexing capability, the functional field test is not a sensitive measure of eye structure, which would be highly useful in the early diagnoses of such eye diseases before substantial degradation has occurred. Such structural tests include, for example, retinal image testing and optical coherence tomography (OCT).

Retinal image testing can be performed with conventional optical imaging methodology and has been routinely used in clinics for retinal structure change evaluation in addition to visual field tests. Devices such as a fundus camera or an indirect opthalmoscope are routinely used for such testing. The retinal image provides valuable information that clinicians can utilize to diagnosis eye diseases. However, only qualitative interpretation of eye structure changes from the retinal photographs can be observed by highly experienced clinicians.

Accordingly, OCT has been used for non-invasive human eye retinal imaging. The cross sectional retinal image provided by an OCT system may allow a clinician to quantitatively evaluate the retinal nerve layer and retinal thickness. Thus, the OCT system may provide valuable clinical information that can be used for early diagnosis of eye diseases, such as macular degeneration or Glaucoma. However, at the end stage of the disease, when, for example, the patients lose most of their Retinal Nerve Fiber Layer (RNFL), the OCT imaging method may have certain technical difficulties in accurately measuring the RNFL. Therefore, it may be difficult to follow the progression of diseases, such as glaucoma, with standard OCT techniques.

A new class of OCT has been developed, known generically as Fourier Domain OCT (FDOCT) or specifically as Spectral Domain OCT (SDOCT) that offers an ability to overcome some of the limitations of standard OCT. SDOCT acquires backscattered optical signatures in an interferometer as a function of wavelength, and through Fourier transform techniques transforms the acquired spectral interferogram into a depth resolved image. Certain advantages of SDOCT over OCT include a higher optical signal to noise ratio, faster imaging speed, and higher axial resolution. These advantages of SDOCT may be applied to ophthalmic imaging to identify small variances in eye structure for early detection of disease, and more refined tracking of disease progression. Descriptions of SDOCT may be found in, for example, Measurement of intraocular distances by backscattering spectral interferometry by Fercher et al., Optical multimode frequency-domain reflectometer by Tan-no et al., and Sensitivity advantage of swept source and Fourier domain optical coherence tomography by Choma et al.

The details of OCT systems used for imaging the human eye are discussed in detail in U.S. Pat. No. 7,140,730 to Jay Wei et al. entitled Optical Apparatus and Method for Comprehensive Eye Diagnosis, the disclosure of which is hereby incorporated herein by reference as if set forth in its entirety. OCT scanners used for imaging the human eye are also discussed in U.S. Pat. No. 6,741,359 to Jay Wei et al. entitled Optical Coherence Tomography Optical Scanner, the disclosure of which is hereby incorporated herein by reference as if set forth in its entirety. Specific SDOCT implementations are also discussed in, for example, United States Patent Publication No. 2007/0291277 to Everett et al. and PCT Publication No. WO2008052793A1. Some limitations of existing SDOCT systems for ophthalmic imaging may include limited field of view, low image brightness, difficulty of subject alignment, and the difficulty and stability of the alignment of multiple optical paths during production.

### SUMMARY OF EMBODIMENTS OF THE INVENTION

Some embodiments of the present invention provide a system for imaging a sample, the system including an optical coherence tomography (OCT) portion having an associated OCT path. A second imaging portion is provided having an associated imaging path, different from the OCT path. This second imaging path may be used for video or digital fundus photography coincident with the OCT imaging. A fixation target having an associated fixation path, different from the second image path and the OCT path is also provided. The OCT path, the second image path and the fixation path share a common conjugate image plane. In other words, the three paths are confocal at a first conjugate system image plane intermediate to the subject and the proximal positions of the OCT path second imaging path, and fixation path.

In further embodiments of the present invention, the common conjugate image plane may be a conjugate plane to each of the OCT image plane, the second image plane and the fixation image plane.

In still further embodiments of the present invention, an OCT beam associated with the OCT path may be telecentric into and out of the common image plane.

In some embodiments of the present invention, the OCT path, the second image path and the fixation path may be separated by first and second dichroic filters.

In further embodiments of the present invention, the sample may be an eye and the system may provide a beam at the front of the eye having a diameter of from about 1.8 to about 2.5 mm.

In still further embodiments of the present invention, the sample may be an eye, the OCT path may include a scanner, a scanned beam, and a pivot point of the scanner may be coincident with a pupil of the eye.

In some embodiments of the present invention, the second imaging path may include an illumination portion and an image capture portion. The system may further include an illumination path including a light source having a range of from about 450 to about 720 nm. The light source may be an light emitting diode (LED). The second image path may include a display and less than about 100 microwatts incident on the sample produces a clear image on the display. The fixation path may include a lens and wherein the lens is designed to image an image plane onto a height of the display.

In further embodiments of the present invention, a beamsplitter may be provided between the second image capture path and illumination path to separate the functions of image capture from image illumination within the second image path. The image capture path and the illumination path may be orthogonally polarized. The illumination path may include an obscuration, and the obscuration may be designed to present a ring, or cone, of illumination to the front of the sample, providing nominally uniform illumination at the sample image plane.

In still further embodiments, the system may provide a ring illumination having an annulus with a diameter of from about 2.5 to about 3.5 mm.

In some embodiments, the sample may be an eye, the system may include an objective lens adjacent the sample and the OCT path, the second image path and the fixation path can all be adjusted in unison to bring each path to a common focus at the target sample image by adjusting an objective lens between the sample and the first conjugate system image plane.

In further embodiments of the present invention, the sample may be an eye and a retina of the eye can be scanned using the system without dilatation of the eye before the scan.

In still further embodiments of the present invention, the system may be miniaturized to provide a handheld system for imaging a sample. The system may further include an objective lens adjacent the sample and the objective lens may have a manual translation capability to accommodate about a +/−20 diopter range.

In some embodiments of the present invention, an adapter configured to adapt a posterior imaging system to operate as an anterior imaging system or an anterior imaging system to operate as a posterior imaging system is provided.

Further embodiments of the present invention provide a handheld system for imaging a sample, the system including an optical coherence tomography (OCT) portion having an associated OCT path. A second imaging portion is provided having an associated second image path, different from the OCT path. A fixation target is provided having an associated fixation path, different from the second image path and the OCT path. The OCT path, the second image path and the fixation path are confocal at a common conjugate system image plane.

In still further embodiments of the present invention, an adapter configured to adapt a posterior imaging system to operate as an anterior imaging system or an anterior imaging system to operate as a posterior imaging system is provided.

### BRIEF DESCRIPTION OF THE DRAWINGS

FIG. 1 is a schematic block diagram illustrating a clinical retinal scanner in accordance with some embodiments of the present invention.

FIG. 2 is a schematic block diagram illustrating handheld SDOCT imagers in accordance with some embodiments of the present invention.

FIG. 3 is a perspective view illustrating an objective lens drive mechanism according to some embodiments of the present invention.

FIG. 4 is a perspective view illustrating an objective lens cell including a built in field stop as part of the lens retention ring according to some embodiments of the present invention.

FIG. 5 is a block diagram of a data processing system that can be used to interface with integrated OCT systems according to some embodiments of the present invention.

### DETAILED DESCRIPTION OF EMBODIMENTS OF THE INVENTION

The present invention will be described more fully hereinafter with reference to the accompanying figures, in which embodiments of the invention are shown. This invention may, however, be embodied in many alternate forms and should not be construed as limited to the embodiments set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention as defined by the claims. Like numbers refer to like elements throughout the description of the figures.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising,” “includes” and/or “including” when used in this specification, specify the presence of stated features, integers, steps, operations, elements, and/or components, but do not preclude the presence or addition of one or more other features, integers, steps, operations, elements, components, and/or groups thereof. Moreover, when an element is referred to as being “responsive” or “connected” to another element, it can be directly responsive or connected to the other element, or intervening elements may be present. In contrast, when an element is referred to as being “directly responsive” or “directly connected” to another element, there are no intervening elements present. As used herein the term “and/or” includes any and all combinations of one or more of the associated listed items and may be abbreviated as “/”.

Unless otherwise defined, all terms (including technical and scientific terms) used herein have the same meaning as commonly understood by one of ordinary skill in the art to which this invention belongs. It will be further understood that terms used herein should be interpreted as having a meaning that is consistent with their meaning in the context of this specification and the relevant art and will not be interpreted in an idealized or overly formal sense unless expressly so defined herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure. Although some of the diagrams include arrows on communication paths to show a primary direction of communication, it is to be understood that communication may occur in the opposite direction to the depicted arrows.

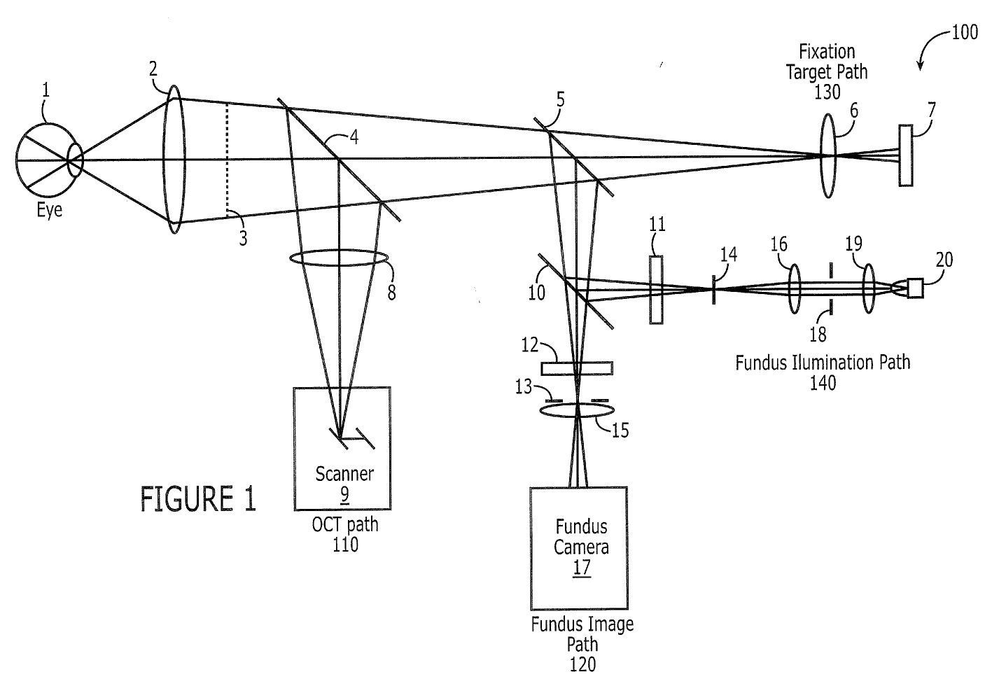

One of the difficulties in using an OCT system to image, for example, the retina of the eye, is that it is difficult to align, i.e. aim, the OCT beam. Thus, according to some embodiments of the present invention a system is provided including an optical coherence tomography system, a fundus camera and a fixation target. According to some embodiments of the present invention, paths to all three of the portions of the system may be adjusted by adjusting an objective lens intermediate to the subject under test and a common conjugate system image plane. Furthermore, some embodiments of the present invention allow scanning of the retina without dilatation of the eye as will be discussed further herein with respect to FIGS. 1 through 4.

Referring first to FIG. 1, a schematic block diagram illustrating a clinical retinal scanner according to some embodiments of the present invention will be discussed. As will be discussed further below with respect to FIG. 1, some embodiments of the present invention provide a spectral domain optical coherence tomography (SDOCT) system 100 configured for retinal diagnostics in the clinical setting. As illustrated in FIG. 1, the system 100 includes an SDOCT portion (OCT path) 110, a fundus (second) imaging portion 120 (Fundus Image Path), and a fixation target 130 (Fixation Path). As further illustrated, these three portions 110, 120 and 130 are combined in the optical system 100 at common conjugate system image plane 3. Furthermore, in some embodiments of the present invention, image plane 3 is a conjugate plane to each of the OCT path 110, the fundus (second) image path 120 and the fixation path 130 independently.

In some embodiments of the present invention, the OCT beam associated with the OCT path 110 may be telecentric into and out of the conjugate image plane 3.

Referring again to FIG. 1, the system further includes a front objective lens 2 adjacent the sample 1, for example, a human eye. In operation, the front objective lens 2 of the system 100 forms from about a 30 to about 45 degree image of the sample (retina 1) and the image is relayed into four paths with beamsplitters. The four paths are the three paths discussed above 110, 120 and 130 and a fundus illumination path 140 illustrated in FIG. 1. The objective lens 2 has translation capability to accommodate about a +/−20 diopter range. In some embodiments of the present invention, the working distance from the last element of the objective lens to the subject eye may be about 15 mm to about 30 mm.

As further illustrated in FIG. 1, the SDOCT portion (OCT path) 110 includes a shortpass filter 4, for example, a 750 nm shortpass filter, a lens 8 and a scanner 9. The scanner may be a 2D scanner. In some embodiments of the present invention, the shortpass filter 4 may be configured to reflect wavelengths from about 750 nm to about 925 nm and transmit wavelengths below about 750 nm for the fixation 130 and fundus 120 paths, which will be discussed further below. The OCT path 110 further includes a 3 mm diameter collimated beam that is incident on the scanner 9 with the lens 8 to form an image of the fiber at the common image plane 3. In some embodiments of the present invention, the combined focal lengths of the front objective lens 2 and the OCT lens 8 are designed to deliver from about 1.8 to about 2.5 mm diameter collimated beam on the cornea. This may yield a 16 micron diameter Gaussian spot on the retina 1 (sample). The pivot point of the scanner 9 and the pupil on the eye 1 are coincident image points, such that scanning through the non-mydriatic eye may be achieved in accordance with some embodiments of the present invention. As used herein, “non-mydriatic” refers to an eye that has not been treated with a drug to, for example, open the pupil. Thus, according to some embodiments of the present invention, the eye does not require dilation.

The fundus (second) imaging path 120 may be configured for alignment and documenting the SDOCT scan. A light source 20, for example, a 700 nm light emitting diode (LED), is used for illumination providing patient comfort and a clear view of the display 7, for example, a color microdisplay, for fixation. As further illustrated in FIG. 1, the fundus path 120 may further include a fundus camera (CCD) 17. Less than about 100 microwatts incident on the cornea may be sufficient to provide a clear image of the retina 1 on the CCD 17. In some embodiments of the present invention, the CCD 17 may be a near infrared (NIR) enhanced Sony ⅓″ CCD. In some embodiments of the present invention, glare may be reduced or possibly eliminated using crossed polarizers 11,12 with a polarization beamsplitter 10 to coalign the illumination and imaging paths. The polarization beamsplitter 10 may be a plate beamsplitter. The fundus (second) image path 120 and the fundus illumination path 140 may be orthogonally polarized without departing from the scope of the present invention.

Ring illumination may be used to reduce or possibly eliminate glare from the cornea. The birefringence of the cornea may cause glare in a crossed polarized imager so a ring illumination may still be needed. The obscuration 14 for the illuminator and aperture stop 13 of the imaging lens are coincident on the subjects pupil 1. The effective pupil size for imaging has about a 2 mm diameter and the ring illumination has an annulus with diameters of from about 2.5 mm to about 3.5 mm. The aperture 18 in the illumination path 140 is imaged onto the retina 1 to provide uniform illumination.

As further illustrated in FIG. 1, the fixation path 130 includes the display 7 to provide the subject a means of fixation for positioning the retina area of interest for the SDOCT scan. In some embodiments of the present invention, the display 7 may be a high resolution color SVGA organic LED (OLED) microdisplay 7. A 650 nm shortpass filter 5 reflects the visible spectrum to the display 7 and transmits from about 680 nm to about 720 nm to the fundus camera (CCD) 17. The lens 6 for the fixation path is designed to image the common image plane 3 onto the height of the display. In some embodiments of the present invention, the display 7 may be a 9 mm display. Alternatively, shortpass filter 5 may be configured for a shorter wavelength, for example 500 nm, and the fixation and fundus illumination wavebands may be reversed, such that the fundus illumination occurs at a shorter wavelength, for example between 450 nm to 500 nm, than reserved for the fixation target.

In some embodiments of the present invention, shortpass filters 4 and 5 may be provided by dichroic filters used to separate the paths as illustrated in FIG. 1. A dichroic filter or thin-film filter is a very accurate color filter used to selectively pass light of a small range of colors while reflecting other colors. However, it will be understood that embodiments of the present invention are not limited to this configuration.

It will be understood that the display 7 may provide static or dynamic images for the subject to view as a fixation target. In some embodiments of the present invention, the display 7 may provide movies for patient comfort, for example, cartoons may be shown for children. The display 7 may also be used to provide an excitation signal synchronized with the scanning system without departing from the scope of the present invention.

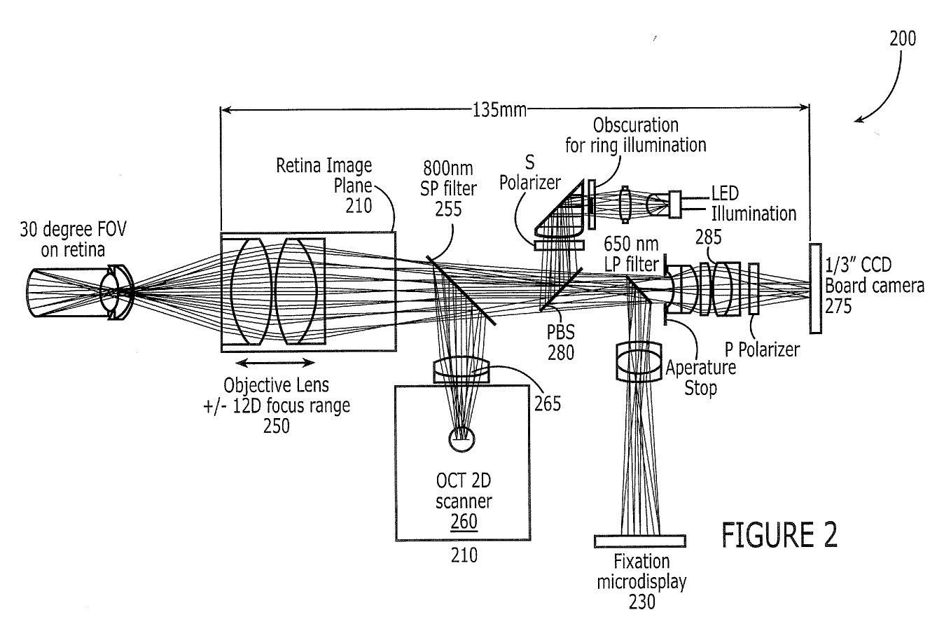

Referring now to FIG. 2, a schematic block diagram of handheld SDOCT system 200 in accordance with some embodiments of the present invention will be discussed. Portable OCT devices are discussed in commonly assigned U.S. patent application Ser. No. 11/535,663, filed Sep. 27, 2006, entitled Portable Optical Coherence Tomography (OCT) Devices and Related Systems, the disclosure of which is hereby incorporated herein by reference as if set forth in its entirety herein. In some embodiments of the present invention, the handheld system 200 may be designed for pediatric patients. As illustrated in FIG. 2, the system 200 includes three primary portions, the SDOCT portion 210, the fundus (second) imaging portion 220, and the fixation target 230. These functionalities (portions) are designed to be integrated in a handheld instrument. The front objective lens 250 forms from about a 30 to about 45 degree image of the retina and then the image is relayed into four paths with beamsplitters. In some embodiments of the present invention, the objective lens 250 may have manual translation capability to accommodate about a +/−20 diopter range and the working distance to the subject eye may be about 20 mm. In some embodiments, the working distance may be shortened to be between about 5.0 mm and about 15 mm, providing a wider accessible field of view. In further embodiments, the objective lens may be configured to operate in contact with the cornea, for a 0.0 mm working distance.

As illustrated in FIG. 2, the OCT path 220 includes a shortpass filter 255, for example, an 750 nm shortpass filter. The shortpass filter 255 may be used to reflect wavelengths from about 750 nm to about 925 nm and transmit wavelengths below about 750 nm for the fixation 230 and fundus paths 220. In some embodiments of the present invention, the OCT path 210 may use a 3 mm diameter fiber collimator that is incident on the 2D scanner 260 with a lens 265 to form an image of the fiber at the image plane 270. The combined focal lengths of the objective lens 250 and the OCT lens 265 are configured to deliver a 2 mm diameter collimated beam on the cornea. This may yield a 16 micron diameter Gaussian spot on the retina. The pivot point of the scanner 260 and the pupil on the eye are coincident image points so the scanning through the non-mydriatic eye may be achieved.

The fundus (second) imaging is used for alignment and documenting the SDOCT scan. A light source, for example, 700 nm LED, is used for illumination providing patient comfort and a clear view of the display, for example, a color microdisplay, for fixation. Less than 100 microwatts incident on the cornea is sufficient to provide a clear image of the fundus on the CCD 275. In some embodiments of the present invention, the CCD 275 used may be an NIR enhanced Sony ⅓″ CCD. In some embodiments of the present invention, glare may be reduced or possibly eliminated using cross polarization with a polarization beamsplitter (PBS) 280 to coalign the illumination and imaging paths. Ring illumination may be used to reduce or eliminate glare from the cornea. The birefringence of the cornea causes glare in a cross polarized imager so a ring illumination may still be needed. The obscuration for the illuminator and aperture stop of the imaging lens are coincident on the subject pupil. In some embodiments of the present invention, the effective pupil size for imaging has about a 2 mm diameter and the ring illumination is an annulus with diameters of about 2.5 mm to about 3.5 mm.

In some embodiments of the present invention, the fixation path 230 uses a high resolution color SVGA OLED microdisplay to provide the subject a means of fixation for positioning the retina area of interest for the SDOCT scan. A 650 nm longpass filter 285 reflects the visible spectrum to the microdisplay 290 and transmits 680 nm to 720 nm to the fundus camera 275. The lens for the fixation path is designed to image the image plane into the height of the display. In some embodiments, the display may have a height of about 9 mm. In some embodiments, the shortpass filter 285 may be configured for a shorter wavelength, for example, about 500 nm, and the fixation and fundus illumination wavebands may be reversed, such that the fundus illumination occurs at a shorter wavelength than reserved for the fixation target.

Thus, some embodiments of the present invention illustrated in FIG. 2 provide an optical instrument for a miniaturized but fully featured SDOCT interface that can be used, for example, for pediatric imaging. As discussed, the system may include a high-speed OCT scanning optics suitable for 2D and 3D retinal imaging; a high-quality non-mydriatic video fundus camera with uniform infrared illumination; an internal fixation target capable of displaying video sequences synchronized with OCT imaging; and a diopter-calibrated focus control capable of +/−20 diopter adjustment. The dipoter control may be manual or automated without departing from the scope of the present invention.

It will be understood that the use of an LED as discussed with respect to some of the embodiments herein may provide a light source that does not produce as much heat and, therefore, reduces the need for a fan. Furthermore, LEDs are more directional than conventional lighting mechanisms. The use of an OLED display for fixation may provide a brighter display than a conventional LED display as well as a higher resolution. In some embodiments of the present invention, the display may be integrated with handheld embodiments of the present invention.

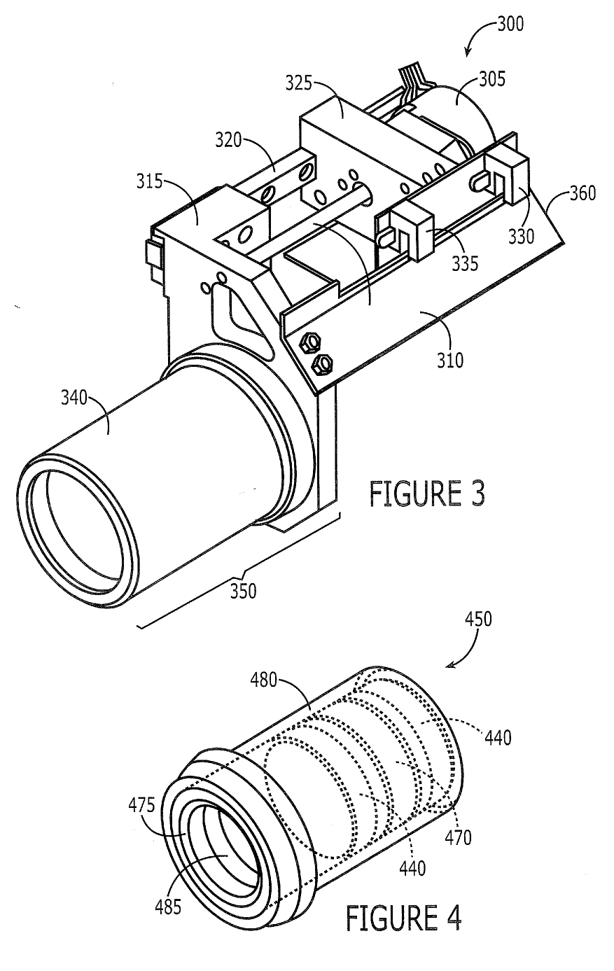

In some embodiments of the present invention, the objective lens may be driven by an objective lens driving mechanism, the details of which will now be discussed with respect to FIGS. 3 and 4. As illustrated in FIG. 3, the objective lens drive mechanism 300 of FIG. 3 includes a stepper motor 305, optical switches 330, a flag 335, a leadscrew 310, an objective lens 340, an objective lens carriage 315, a linear slide bearing 320 and a motor mount 325. In particular, the objective lens drive mechanism 300 according to some embodiments of the present invention includes an integrated stepper motor 305 with a free moving leadscrew combination 310 affixed to the objective lens carriage 315, which in turn is affixed to a linear slide bearing 320. This creates a complete motion control system with a minimum of components as listed: Stepper motor/leadscrew 305/310, Objective Lens Carriage 315, Linear Slide Bearing 320 and Stepper Motor Mount 325 as illustrated in FIG. 3. In some embodiments of the present invention, the assembly has a default position resolution of 1/20 of a diopter. As discussed above, the Objective Lens Drive Mechanism also includes a set of optical switches 330 and flag 335 used to control the travel range of the mechanism. In some embodiments of the present invention, the objective lens cell 350 is removable from the Objective Lens Carriage 360, for example, via a standard screw thread interface.

Referring now to FIG. 4, a detached objective lens cell 450 will be discussed. As illustrated in FIG. 4, the objective lens cell 450 includes first and second objective lenses 440, an objective lens spacer 470, a field stop 475, an objective lens housing 480, and a lens retaining ring 485. As illustrated, the Objective Lens Cell 450 includes a built in field stop 475 as part of the lens retaining ring 485.

It will be understood that details of the systems 100 and 200 discussed herein with respect to FIGS. 1 through 4 are provided herein for exemplary purposes only and, therefore, embodiments of the present invention are not limited to the configuration discussed herein. For example, the position of the long pass and short pass filters may be changed without departing from the scope of the present invention.

As discussed above, embodiments of the present invention provide retinal scanners. Retinal scanners are used for posterior imaging of the retina and may be referred to generally as posterior imaging systems. Anterior imaging systems may be used for anterior imaging of the cornea and the anterior chamber. Typically, posterior imaging systems for imaging the posterior region of the eye and anterior imaging systems for imaging anterior segments of the eye require different penetration depths, imaging depths, focal positions, and/or scanning optics. Thus, posterior imaging systems developed for high quality posterior imaging applications have not been applicable to high quality anterior applications. Similarly, anterior imaging systems developed for high quality anterior imaging applications have not been applicable to high quality posterior applications. However, it will be understood that an adapter may be positioned in the posterior or anterior system according to some embodiments of the present invention so as to allow the posterior system to operate as an anterior system or the anterior system to operate as a posterior system. Details with respect to adapters are discussed in commonly assigned copending U.S. patent application Ser. No. 11/930,865, filed on Oct. 31, 2007 entitled Optical Coherence Imaging Systems Having a Mechanism for Shifting Focus and Scanning Modality and Related Adapters, the disclosure of which is hereby incorporated herein by reference as if set forth in its entirety.

As discussed above, in some embodiments of the present invention, the illumination path may be used for illuminating the fundus of the eye, and the illumination path may include a light source having a range of from about 500 to about 720 nm. In certain embodiments, the light source may be a LED. In some embodiments, as discussed herein, the second (fundus) image path may include a video or digital capture device for capturing the image of subject and a display for displaying the captured image. Less than about 100 microwatts incident on the sample through the illumination path may produce a clear image on the display. The fixation path may include a lens and the lens is designed to image a display as an object of the fixation path onto the subject plane. The fixation field of view may be nominally equivalent to the second imaging path field of view. Each of the OCT path, second imaging path, and fixation path share a common focus on the subject plane.

As further discussed above, in some embodiments of the present invention, the second image path is a path for imaging the fundus of the eye, the path including further bifurcation into an illumination path and an image capture path, the two bifurcated paths being orthogonally polarized, and the illumination path including an obscuration to create a ring of illumination that reduces specular reflections from the front surface of the sample, while nominally uniformly illuminating the fundus of the sample.

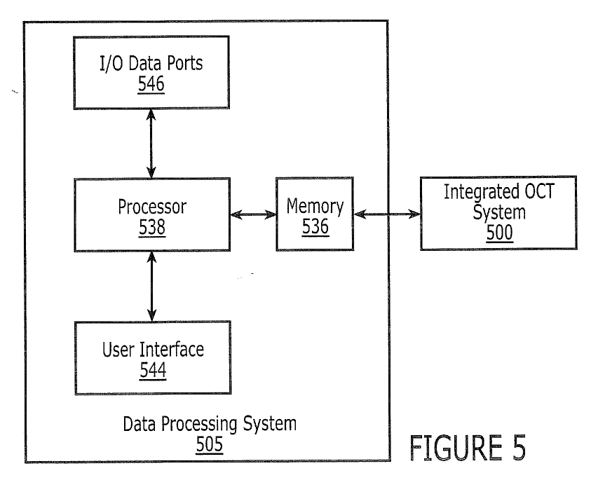

It will be understood that the product or intermediate products of the system discussed above in accordance with some embodiments of the present invention may be processed using a data processing system contained in, for example, a computer. As illustrated in FIG. 5, outputs of integrated OCT systems 500 in accordance with some embodiments of the present invention may be provided to a data processing system 505 and processes to provide meaningful information. As illustrated in FIG. 5, an exemplary embodiment of a data processing system 505 suitable for use with systems in accordance with some embodiments of the present invention will be discussed. The data processing system 505 typically includes a user interface 544, such as a keyboard, keypad, touchpad or the like, I/O data ports 546 and a memory 536 that communicate with a processor 538. The I/O data ports 546 can be used to transfer information between the data processing system 505 and another computer system or a network. These components may be conventional components, such as those used in many conventional data processing systems, which may be configured to operate as described herein.

Example embodiments are described above with reference to block diagrams and/or flowchart illustrations of methods, devices, systems and/or computer program products. It is understood that a block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means (functionality) and/or structure for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the functions/acts specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, example embodiments may be implemented in hardware and/or in software (including firmware, resident software, micro-code, etc.). Furthermore, example embodiments may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Computer program code for carrying out operations of data processing systems discussed herein may be written in a high-level programming language, such as Java, AJAX (Asynchronous JavaScript), C, and/or C++, for development convenience. In addition, computer program code for carrying out operations of example embodiments may also be written in other programming languages, such as, but not limited to, interpreted languages. Some modules or routines may be written in assembly language or even micro-code to enhance performance and/or memory usage. However, embodiments are not limited to a particular programming language. It will be further appreciated that the functionality of any or all of the program modules may also be implemented using discrete hardware components, one or more application specific integrated circuits (ASICs), or a programmed digital signal processor or microcontroller.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated.

In the drawings and specification, there have been disclosed exemplary embodiments of the invention. However, many variations and modifications can be made to these embodiments without substantially departing from the principles of the present invention. Accordingly, although specific terms are used, they are used in a generic and descriptive sense only and not for purposes of limitation, the scope of the invention being defined by the following claims.

That which is claimed is:

1. A system for imaging a sample, the system comprising:
an optical coherence tomography (OCT) portion having an associated OCT path;
a second imaging portion having an associated second image path, different from the OCT path; and
a fixation target having an associated fixation path, different from the second image path and the OCT path, wherein the OCT path, the second image path and the fixation path share a common conjugate image plane.

2. The system of claim 1, wherein the common conjugate image plane is a conjugate plane to each of the OCT path, the second image path and the fixation path.

3. The system of claim 1, wherein an OCT beam associated with the OCT path is telecentric into and out of the common image plane.

4. The system of claim 1, wherein the OCT path, the second image path and the fixation path are separated by first and second dichroic filters.

5. The system of claim 1, wherein the sample is an eye and wherein the system provides a beam at the front of the eye having a diameter of from about 1.8 to about 2.5 mm.

6. The system of claim 1:
wherein the sample is an eye;
wherein the OCT path includes a scanner; and
wherein a pivot point of the scanner is coincident with a pupil of the eye.

7. The system of claim 1, wherein the second imaging path includes an illumination portion and an image capture portion, and further comprising an illumination path including a light source having a range of from about 450 to about 720 nm.

8. The system of claim 7, wherein the light source comprises a light emitting diode (LED).

9. The system of claim 7, wherein the second image path includes a display and wherein less than about 100 microwatts incident on the sample produces a clear image on the display.

10. The system of claim 9, wherein the fixation path includes a lens and wherein the lens is designed to image an image plane onto a height of the display.

11. The system of claim 7, further comprising a beamsplitter between the second image path and the fundus illumination path to separate the second image path and the second image path, wherein the second image path and the fundus illumination path are orthogonally polarized.

12. The system of claim 7, wherein the fundus illumination path includes an obscuration.

13. The system of claim 1, wherein the system provides a ring illumination having an annulus with a diameter of from about 2.5 to about 3.5 mm.

14. The system of claim 1:
wherein the sample is an eye;
wherein the system further comprises an objective lens adjacent the sample; and
wherein the OCT path, the second image path and the fixation path can all be adjusted by adjusting the objective lens at a retinal image plane of the eye.

15. The system of claim 11:
wherein the sample is an eye; and
wherein a retina of the eye can be scanned using the system without dilating the eye before the scan.

16. The system of claim 1, where the system is miniaturized to provide a handheld system for imaging a sample.

17. The system of claim 16, wherein the system further comprises an objective lens adjacent the sample and wherein the objective lens has a manual translation capability to accommodate about a +/−20 diopter range.

18. The system of claim 1, further comprising an adapter configured to adapt a posterior imaging system to operate as an anterior imaging system or an anterior imaging system to operate as a posterior imaging system.

19. A handheld system for imaging a sample, the system comprising:
an optical coherence tomography (OCT) portion having an associated OCT path;
a second imaging portion having an associated second image path, different from the OCT path; and
a fixation target having an associated fixation path, different from the second image path and the OCT path, wherein the OCT path, the second image path and the fixation path are confocal at a common image plane.

20. The handheld system of claim 19, further comprising an adapter configured to adapt a posterior imaging system to operate as an anterior imaging system or an anterior imaging system to operate as a posterior imaging system.
